SEQUENCE LISTING (7 sequences)

SEQ ID NO: 1 - DNA, 6 bp, Artificial Sequence
  Synthetic sequence
actgct

SEQ ID NO: 2 - DNA, 29 bp, Artificial Sequence
  Synthetic sequence
ccgtgcagatcttaatgtgccagtaaaag

SEQ ID NO: 3 - DNA, 20 bp, Artificial Sequence
  Synthetic primer
ccgtgcagatcttaatgtgc

SEQ ID NO: 4 - DNA, 19 bp, Artificial Sequence
  Synthetic primer
gcgctgtaccaaaggcatc

SEQ ID NO: 5 - DNA, 43 bp, Enterococcous faecalis
ccgatgccttagtttcaagtggtgcgattgacatcgttgtcat

SEQ ID NO: 6 - DNA, 45 bp, Artificial Sequence
  Synthetic sequence
tggaccagaccagctatgggggagctggggaaggtgggaatgtga

SEQ ID NO: 7 - DNA, 23 bp, Artificial Sequence
  Synthetic sequence
cacgtatgggggagctggggtat

(19) United States
(12) Patent Application Publication
(10) Pub. No.: US 2011/0059859 A1
(43) Pub. Date: Mar. 10, 2011

(54) Molecule Detecting System

(76) Inventors: Zhiping Liu, Beijing (CN); Sheng Luan, Berkeley, CA (US)

(21) Appl. No.: 12/991,825

(22) PCT Filed: May 11, 2009
(86) PCT No.: PCT/CN09/00507

§ 371 Date: Nov. 9, 2010

Related U.S. Application Data
(60) Provisional application No. 61/126,929, filed on May 9, 2008.

(51) Int. Cl. C40B 30/04; C07H 21/02; C08B 31/00; C08B 37/06; C08B 37/02; C07K 14/00; C40B 40/06; C40B 40/10; C40B 40/12; C07H 21/04

(52) U.S. Cl. 506/9; 536/23.1; 536/102; 536/2; 536/112; 530/350; 506/16; 506/18; 506/19

(57) ABSTRACT

The invention disclosed here has several key elements: direct probe-on-sensor detection, mesh probe, and mesh reporter. Furthermore, the mesh probe and reporter shall have some physical cross-linking between the polymer backbone(s), either covalently or non-covalently. In combination, this invention enables one to build an ultra-sensitive, low-cost, and highly portable system for molecular detection. Among its many potential applications is a direct detection of nucleic acids in crude lysate in a multiplex format, without the requirements for nucleic acids extraction and amplification. Such an improvement is likely to change the practice of genotyping and gene expression profiling done in a clinic setting. Another application is an ultrasensitive ELIZA assay in a multiplex format.

### TECHNICAL FIELD

This invention relates to a molecule detecting system. More specifically, this invention relates to mesh probes which are used to form an array for a biosensor employing optical detection of analytes.

### BACKGROUND OF THE INVENTION

Several terms used in this disclosure are defined as follows:

Meshed polymer is a polymer derivative of which one or more chemical or biological species are cross-linked to the polymer backbone.

Mesh Probe is a derivatized polymer in which the cross-linked chemical or biological molecules are capture molecules whose function is to selectively and specifically bind to target molecules.

Mesh Reporter is a derivatized polymer that contains a recognition species and signaling species. Both can be either chemical or biological in nature and are cross-linked to the same polymer backbone, hence are physically linked together. The function of the recognition molecule is to selectively and specifically binds to a “tag” that is usually associated with target molecules. The function of the signaling molecules is to generate controlled release of the signals for detection, either directly or through a subsequent event, such as fluorescence or chemiluminescence.

Bridging Molecule is a cross-linker that introduces intra and inter-molecular cross-linking on the polymer backbones. It is usually a bi-functional cross-linker whose functional groups only reacts with the polymer backbone or the derivative groups introduced on it. There are exception to this general rule. An example is a G-rich oligonucleotide with a reactive group at one end. So long this end is cross-linked to the polymer backbone, the subsequent Hoogsteen base-paring among the oligonucleotide shall generate a non-covalent “cross-linking” (or interconnects) either within or between the polymers.

Biomolecules and other analytes can be detected using arrays of selective or specific probes which bind target analytes. Schemes have been developed in biosensor array technology to arrange probe spots on substrates or biochips. For example, a variety of schemes are described in M. Schena and R. W. Davis, DNA Microarrays: A Practical Approach (M. Schena ed., Oxford University Press 1999). Arrays are used to detect and discover gene sequences, to select and test drug molecule candidates, to investigate toxicological or pharmacological action, and other uses. Targets may bind to probes of an array through a variety of interactions, including nucleic acid base pairing or hybridization, protein-protein interactions, protein-ligand interactions, enzyme-substrate interactions, receptor-ligand interactions, and other chemical reactions.

Biosensors allow simultaneous examination of a large number of interactions between biomolecules, such as proteins or nucleic acids, in a microarray format. They represent a powerful tool in utilizing the large amount of sequence information generated from the Human Genome Project, as well as that from genome sequencing of other organisms.

The signal from analyte species is generally small, and background arising from various sources makes the signal-to-noise ratio of the measurement relatively low. Low signal translates to low signal-to-noise ratio and poor detection of analytes. A solution is to enhance the signal from analytes, to increase the inherent signal-to-noise ratio of the detection. Increasing the signal-to-noise ratio lowers the detection limit for analytes, making it possible to observe analytes at lower concentration, opening new doors for applications involving molecular detections.

### SUMMARY OF THE INVENTION

To solve above-mentioned problems, applicant provides following technical solutions:

The present application provides a kind of molecule detecting system, wherein the said system comprising:

an optical sensor;

an array of capture molecules which are attached on the sensor surface through cross linking to a polymer that has limited interconnects amongst the polymer chains.

Wherein said array of capture molecules are attached on the surface of a thin transparent solid or porous substrate through cross linking to a polymer that has limited interconnects amongst the polymer chains, and the said transparent substrate is fixed above the surface of an optical sensor.

Wherein said polymer is a natural polymer.

Wherein said natural polymer is a linear or branched polysaccharide.

Wherein said linear polysaccharide is dextran.

Wherein said branched polymer is a glycogen or amylopectin.

Wherein said polymer is branched-DNA.

Wherein said polymer is a hydrogel.

Wherein said capture molecule is a biomolecule.

Wherein said biomolecule is a protein.

Wherein said biomolecule is a DNA of RNA.

Wherein said capture molecule of claim is a PNA or LNA.

Wherein said polymer is a synthetic polymer.

Wherein said synthetic polymer is Poly(methyl vinyl ether-alt-maleic anhydride)

Wherein said interconnects amongst the polymer chains is covalent formed by cross-linking with a bi-functional cross-linker.

Wherein said bi-functional cross-linker is 4,7,10-Trioxa-1,13-tridecanediamine.

Wherein said interconnect amongst is form by non-covalent interactions.

Wherein said non-covalent interaction is a four stranded DNA or RNA structure formed by Hoogsteen base-pairing between G-rich oligonucleotides cross-linked to the polymer.

Wherein said G-rich oligonucleotide is Q3 derivatized at the end with a functional group for cross-linking to the polymer.

The present application also provides a method of making polymer used in molecule detecting system comprising following steps:
a) reacting a polysaccharide with sodium periodate, thereby forming a linear or branched polymer having a large number of aldehyde groups;
b) adding a molecule having or derivatized with a reactive amino groups; and
c) complete the coupling reaction in the presence of NaCNBrBH3,
wherein the said polymer has limited interconnects amongst the polymer chains.

Wherein a reporter molecule of which both the signaling molecules and at least a recognition molecule are cross-linked to the polymer prepared.

Wherein said reporter which the signaling molecule is either horse reddish peroxidase or alkaline phosphotase and the recognition molecule is straptavidin.

The present application provides a method for analyte detection comprising:
a) contacting an analyte sample with the array of molecules of claim 1;
b) then contact the array of molecule with the reporter of claim 21;
c) adding to the array a chemiluminescent substrate;
d) retrieve the data directly from the optical sensor without the use of an external scanner.

Wherein said analyte is a nucleic acid, and the analyte sample has not been subject to any enzymatic amplification.

An array of capture molecules for use in detecting analytes on an optical sensor, where the capture molecules are attached on the surface of a thin transparent solid or porous substrate through cross linking to a polymer that has limited interconnects amongst the polymer chains, and the said transparent substrate is fixed above the surface of an optical sensor.

### BRIEF DESCRIPTION OF THE DRAWINGS

FIG. 1 illustrates an embodiment of a low-light image sensor enclosure attached to an embodiment of a reading station.

FIG. 2 illustrates a side view of an embodiment of a low-light image sensor enclosure.

FIG. 3 illustrates fluorescence detection of a probe spot using a CMOS image sensor.

FIG. 4 illustrates a biosensor system for detecting analytes and displaying analyte array data.

FIG. 5a illustrates an AFP detection signal.

FIG. 5b illustrates a PSA detection signal FIG. 5c illustrates AFP Cleft) and PSA detection signal together.

### DETAILED DESCRIPTION OF THE INVENTION

### Mesh Probes

In general, the probes of an array capture and bind the analyte to be detected. Probe capture or target-binding moieties include nucleic acids, polynucleotides, proteins, peptide nucleic acids, small molecules, and a wide variety of biomolecules.

Target-binding probe moieties include antibodies. In one example, streptavadin is used to detect a biotin labeled molecule.

In one aspect, this invention embodies increased numbers of analytes within each point or spot of the array. The increased number of analytes per spot is achieved by compositions of mesh probes which can capture enhanced numbers of analyte moieties.

In one embodiment, meshed polymers are formed from a polymer linked to chemical or biomolecules, where the chemical or biomolecules include a probe or probes, thereby forming a mesh probes. The chemical or biomolecules containing the probe or probes are coupled to the polymer by covalent bonds, or by non-covalent chemical interactions such as ionic interactions or weak binding forces. The polymer may be a linear or branched polymer, such as a linear or branched polysaccharide or oligonucleotide, for example.

The polymer can be a solid, gel or amorphous composition, in the form of layers, beads, discs or mixtures thereof, and can be homogeneous or heterogeneous, linear or branched, side-chain branched, branched comb, or star or dendrimeric. Polymer branches may be long-chain branches or short-chain branches. The polymers are made by synthetic methods, or may be obtained as natural products isolated from naturally-occurring sources. Examples of the polymer include carbohydrates, saccharides, homopolysaccharides, heteropolysaccharides, agarose, amylose, amylopectin, glycogen, dextran, cellulose, chitin, chitosan, peptidoglycan, and glycosaminoglycan. In some embodiments, the polymer is a highly branched dextran. In further embodiments, the polymer is a hydrated dextran or agarose, such as a hydrogel, or a polyacrylamide gel. Further examples of the polymer used to make the meshed polymers include oligonucleotides, peptides, peptide nucleic acids, proteoglycans, glycoproteins, and glycolipids. In further embodiments, the polymer can be an antibody or antibody fragment.

Further examples of polymers useful for making meshed polymers include diol-containing polymers, such as polymers having gem-diol and vicinal-diol groups. Another example is a polymer having a hydroxyl group vicinal to an ester group, such as a phosphodiester linkage in an RNA.

Another example is a polymer having a plurality of hydroxyl groups. Mixtures of any of these polymeric species may be used in an embodiment of this invention.

Synthetic polymers can be used as the backbone of the mesh probe. A example is Poly(methyl vinyl ether-alt-maleic anhydride), whose monomer is:

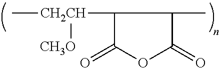

The anhydride group contained therein can readily react with the amine groups in proteins or derivated oligonucleotides forming the “mesh probe”. Another example is Poly[(o-cresyl glycidyl ether)-co-formaldehyde}, whose repeating unit is

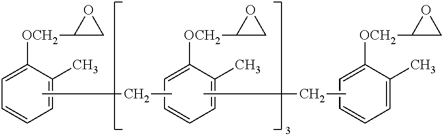

The epoxy groups contained in this polymer can be converted first to adjacent diol groups by the ring-opening in alkaline pH, and then to aldehyde groups by oxidation with NaIO₄. The oxidated polymer can then be coupled to primary amine-containing molecules by reductive amination.

The mesh probes are prepared by coupling the chemical or biomolecule to the polymer. In some embodiments, the mesh probes are prepared by a conjugation reaction of a functional or reactive group on the chemical or biomolecule with the polymer, which couples the chemical or biomolecule to the polymer. Functional or reactive groups on the chemical or biomolecule include, for example, aldehydes, hydroxyls, amines or amino groups, carboxylates, sulfhydryl groups, and mixtures thereof. In one embodiment, avidin-biotin interaction is used for the conjugation reaction.

The mesh probes are prepared by coupling or reacting a chemical or biomolecule with the polymer, where the polymer may be derivatized to contain a plurality of sites for attachment to the functional or reactive groups of the chemical or biomolecules, either directly, or indirectly via linker groups. The derivatized polymer has reactive groups which can be used to attach chemical or biomolecules. The reactive groups of the derivatized polymer may be aldehydes, hydroxyls, amines or amino groups, carboxylates, sulfhydryls, isothiocyanates, N-hydroxysuccinimide esters, ketones, glyoxals, epoxides, oxiranes, imidoesters, carbodiimides, alkylphosphates, anhydrides, maleimides, aziridines, acryloyls, fluorophenyls, diazoacetyls, N-acylimidazoles, succinimidyl carbonates, carboxymethyl groups, isocyanates, hydrazide groups, and mixtures thereof.

The polymer may have a reactive amine group such as the amino group in chitosan. In further embodiments, the polymer has reactive functional groups such as sulfates, carboxylates, or phosphate groups. Examples of sulfate-containing polymers include chondroitin sulfate, dermatan sulfate, heparin sulfate and keratin sulfate. Examples of carboxylate-containing polymers are polysaccharides containing groups which are derivative of sialic acid, aldonic acid, uronic acid, oxoaldonic acid, and ascorbic acid.

Examples of phosphate-containing polymers include DNA or RNA. These polymers may be coupled to a chemical or biomolecule to make a mesh probe using bifunctional linkers such as homobifunctional, heterobifunctional, or multifunctional linkers. For example, the mesh probe may be a polynucleotide polymer coupled to another polynucleotide. In one example, RNA is oxidized to provide aldehyde groups for cross-linking to chemical or biomolecules to make a meshed polymer.

A variety of chemical or biomolecules may be coupled to the polymer to provide mesh probes capable of binding a variety of targets. In other words, a single polymer chain may be coupled to a variety of chemical or biomolecules to provide a mesh probe. Mixtures of mesh probes may be used in an embodiment of this invention.

In one embodiment, to prepare a mesh probe, amino groups on each of the polymer and the chemical or biomolecule are linked using dithiobis(succinimidylpropionate), disuccinimidyl tartarate, or disuccinimidyl glutarate. In further embodiments, a sulfhydryl group of the chemical or biomolecule is linked with an amine group of the polymer using N-succinimidyl 3-(2-pyridyldithio)propionate or m-maleimidobenzoyl-N-hydroxysuccinimide ester. In another embodiment, a sulfhydryl group of the chemical or biomolecule is linked with an aldehyde group of the polymer using 4-(N-maleimidomethyl)cyclohexane-1-carboxyl-hydrazide or 3-(2-pyridyldithio)propionyl hydrazide. In a further embodiment, a sulfhydryl group of the chemical or biomolecule is linked with a carboxylate group of the polymer using 4-(p-azidosalicylamido)butylamine.

Amino groups on each of the polymer and the chemical or biomolecule may be linked in further embodiments using heterobifunctional crosslinkers N-5-azido-2-nitrobenzoyloxysuccinimide or N-hydroxysulfosuccinimidyl-4-azidobenzoate.

In one embodiment, the conjugation is performed by reacting the hydroxyl groups of the polymer with a carbonylating agent such as N,N′-carbonyldiimidazole to form an intermediate imidazolyl carbamate, which in turn, can react with N-nucleophiles such as amines, amino-containing moieties such as peptides and proteins, to give an N-alkyl carbamate linkage.

In another embodiment, the conjugation is performed by reacting the hydroxyl groups of the polymer with N,N′-disuccinimidylcarbonate, followed by reaction with an amino-containing moiety, such as, for example, an amino group on an oligonucleotide. The amino group may be a terminal amino group or proximal to a terminus of the oligonucleotide.

The conjugation may be performed by reacting the polymer with 3-maleimidopropionic acid, followed by reacting the product, a derivatized polymer, with an amino group on an oligonucleotide.

In another embodiment, the conjugation is performed by reacting polymer hydroxyl groups with alkyl halide terminal groups of the chemical or biomolecule to give ether linkages in the mesh probe.

Polymers containing hydroxyl groups on adjacent carbon atoms, for example saccharides or glycoproteins, may be reacted with sodium periodate to produce aldehyde functional groups on the polymer that can be used to couple chemical or biomolecules to prepare mesh probes. Subsequent reaction of the aldehyde functional groups on the polymer with an amine-containing chemical or biomolecule produces a Schiff's base linkage between the polymer and the molecule. The Schiff's base linkage can be reacted with reducing agents such as sodium borohydride or sodium cyanoborohydride to produce a secondary or tertiary amine linkage between the polymer and the chemical or biomolecule.

In further embodiments, mesh probes are prepared using photoreactive crosslinkers. For example, amino groups on each of the polymer and the chemical or biomolecule may be coupled to a photoreactive crosslinker, thereby forming a meshed polymer in which the polymer is coupled to the chemical or biomolecule through a linking group. In one example, an amino group of the polymer can be coupled to N-hydroxysuccinimidyl-4-azidosalicylic acid, and a amino group of the chemical or biomolecule may then be coupled by photolysis to form the derivatized polymer in which the polymer is coupled to the chemical or biomolecule through a linking group.

In another example, the sulfhydryl group of the chemical or biomolecule may be coupled to 1-(p-azidosalicylamido)-4-(iodoacetamido)butane, and an amino group of the polymer may then be coupled by photolysis to form the derivatized polymer in which the polymer is coupled to the chemical or biomolecule through a linking group.

In another example, the aldehyde group of the polymer may be coupled to p-azidobenzoyl hydrazide, and an amino group of the chemical or biomolecule may then be coupled by photolysis to form the derivatized polymer in which the polymer is coupled to the chemical or biomolecule through a linking group.

For the purpose of practical utility, it is desirable or maybe even essential to introduce certain degree of physical linkage, either inter- or intra-molecular, of the polymers themselves. Given the size, the mesh probe itself may subject to backbone breakage by mechanical sheering force that are usually associated with pipetting, stirring etc. This kind of breakage is especially problematic if the polymer backbone is linear form. By introducing some physical linking of the polymer backbone (including intra-molecular linking) will alleviate this problem. The physical linkage can be covalent or non-covalent in nature. Homo-bifunctional cross-linker, such as 4,7,10-Trioxa-1,13-tridecanediamine, can used to generate covalent linkage. Other strong molecular interactions can be exploited for the non-covalent linkage, such as Hoogsteen base-pairing between guanines that leads to four stranded DNA formation. Given the behavior of polymers in aqueous solution that they form a rather compact random-coil, the linking of the backbone from the scheme outlined here will be predominantly intra-molecular, rather than inter-molecular.

The coupling of mesh probes to the surface of the sensor to make an array can be done in a number of ways. For example, the surface may be derivatized with an epoxide, which can react with reactive —OH or —NH₂— groups in the mesh probes. In another example, the sensor surface is treated with poly(lysine), and mesh probes or biomolecules are spotted onto the surface. UV irradiation may optionally be used to crosslink the mesh probes or biomolecules to a substrate, such as a glass slide or passivation layer adjacent to an electronic device. Mesh probes or oligonucleotides may be coupled to a sensor surface which has been derivatized with aldehyde, amine, or isothiocyanide groups. The mechanics for the formation of the array can be spotting, inkjet printing, or direct on-chip synthesis.

Digital Image Biosensor System

In one aspect of this invention, detection of analytes with a biosensor system is enhanced by using digital image or “machine vision” sensing technology which can be used to read out the signal from the analyte bound to the array with less background, and correspondingly higher signal-to-noise ratio. In one embodiment, the biosensor system employs digital image sensing technology including a digital image sensor on a daughterboard, an array of mesh probes, a low-light enclosure for the sensor which may provide thermal cooling for the sensor, and methods of integrating analyte signals.

In one embodiment, optical signal from an array comprising capture species such as mesh probes is detected using a digital image sensor. The digital image sensor includes a matrix of photosensor elements. The mesh probes may be spotted in an array on the digital image sensor and may be linked either covalently or non-covalently to a surface of the digital image sensor. In alternative embodiments, the array is spotted on a glass slide which can be placed adjacent to the digital image sensor. In such embodiments, a fiber optical coupler is optionally located between the glass slide and the sensor.

Yet in another embodiment, the mesh probes are spotted then cross-linked to a very thin transparent support, either porous or solid. An example of the thin transparent solid support is the cover slip for a glass slide. The thin transparent support is then directly attached and fixed onto the surface of the digital image sensor, with the probe side on top. The fixation can be accomplished through a mechanical mechanism or a transparent adhesive. This kind of thin support is very likely to be brittle, hence unlikely substrate for microarray-based applications in practice. However, this potential thin support breakage is circumvented by attaching it directly to the surface of a digital image sensor. In such a scheme, the digital image sensor can be reused by removing the used transparent thin support with a fresh one. For practical utilities, this feature will be very beneficial in cases where the digital image sensor is a part of disposable unit. The use of the “thin” transparent support here is critical. Thick materials or more distance between the sensing elements (pixels) and the light generating source (mesh probe) will compromise the performance of this system, since the light collected by a pixel is inversely proportional to the cubic power of the distance between the pixel and the light source. A regular glass slide would be deemed too thick for such an application.

The localized region of each discrete probe spot in the array may be larger than that of an individual photosensor element in the digital image sensor. Alternatively, the probe spots may be about the same size as an individual photosensor element.

The digital image sensor is, in one embodiment, a CMOS active pixel sensor. Each pixel of the CMOS active pixel sensor includes a photodiode cell which is linked to its own analogue to digital converter, amplifier, and register. The top layer of the CMOS active pixel sensor is a passivation layer, which may be silicon dioxide substantially transparent to light, and serves as a fluid barrier to insulate the semiconductor circuitry from the analyte solution to be delivered to the array.

In one aspect, this invention relates to enhancement of signal from detected species by detection of the signal with a digital image sensor, in which the array is formed directly on the digital image sensor. In one embodiment, optical detection of analyte chemiluminescence emission is performed with an array formed on a thin passivation layer on top of a digital image sensor. In this embodiment, signal is advantageously enhanced by the proximity of the array to the photosensitive elements of the digital image sensor.

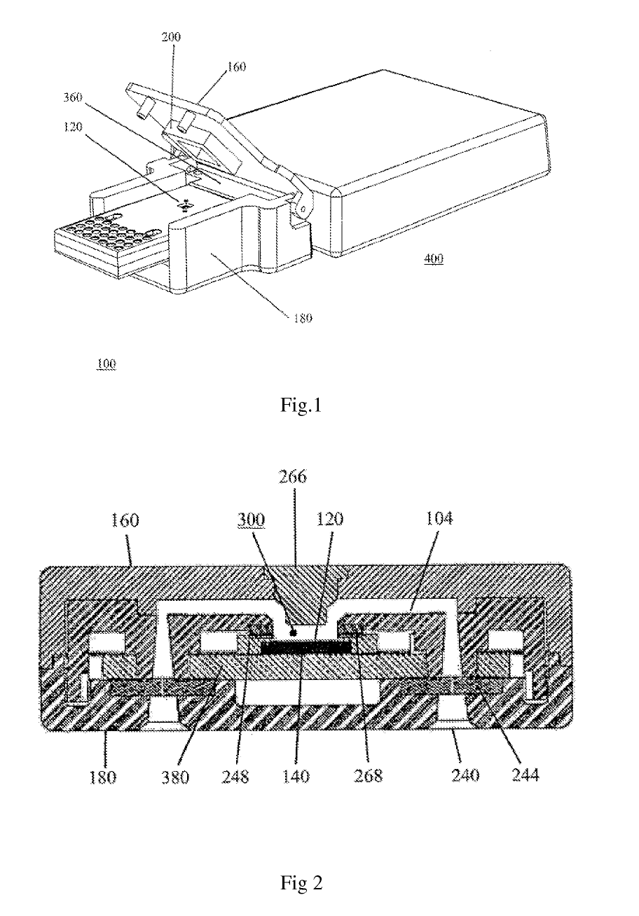

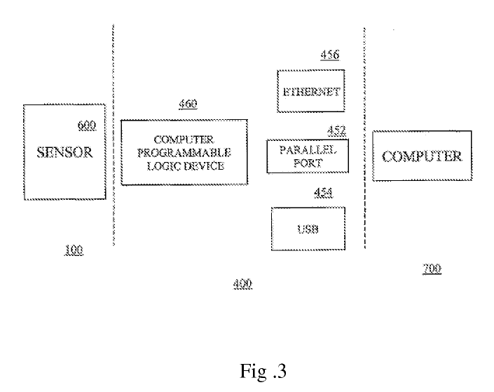

In another aspect of this invention, the biosensor provides increased signal-to-noise ratio of the measurement of analyte array light signals by reducing the background radiation impinging on the image sensor detector. As illustrated in the embodiment of FIG. 1, a low-light enclosure 100 is provided to contain the optical image sensor and the array. The enclosure 100 has a top shell 160 and a bottom shell 180. The bottom shell 180 supports a printed circuit board for the optical image sensor, optional cooling elements for the sensor, and mechanically receives the top shell 160. In the embodiment of FIG. 1, the printed circuit board for the optical image sensor is contained within a second enclosure 150 which is attached to the connector 360. [rewrite from new FIG. 2] The top shell 160, when received by the bottom shell 180, provides a low-light region 300 defined by a barrier 200 which sealingly surrounds the array 120. Fluid contacts the array in the low-light region defined by the barrier 200.

A fluid entry opening 240 is provided in the top shell 160. In operation, fluid is charged to the array 120 by injecting a liquid containing target molecules through the fluid entry opening 240. The fluid pools in the low-light region 300. Optionally, a capillary structure or fluid channel is formed within the enclosure 100 to deliver the analyte from the fluid entry opening 240 to the array 120. The fluid entry opening 240 optionally includes a septum through which fluid is introduced, the septum being a barrier for both fluids and light.

In some embodiments, an optional fluid entry opening 320 is defined by the bottom shell 180. An optional fluid channel 340 connects the optional fluid entry opening 320 to the low-light region 300.

In other embodiments, the barrier 200 is attached to a glass slide bottom window 280, which is adjacent to the image sensor when the top shell is received by the bottom shell. In these embodiments, the fluid pools on the glass slide bottom window 280 and the array is formed on the glass slide bottom window 280 within the low-light region 300.

The enclosure 100 is connected to the reading station 400 so that the array is substantially gravitationally level. Optionally, the enclosure may be attached to the reading station and operated in any gravitational orientation. In these optional embodiments, the fluid entry opening and enclosure may encapsulate the analyte fluid by surface tension and capillary effects in any orientation, to the extent that the analyte array signals may be read out.

As illustrated in the embodiments of FIGS. 1 and 2, the printed circuit board 380 supporting the optical image sensor 140 is electrically connected to the reading station 400 through an electrical connector 360. Optionally, the bottom shell 180 provides opening(s) 220 for accessing the electronic circuits of the optical image sensor 140.

In one embodiment, a biosensor system is provided which integrates analyte signal to increase the signal-to-noise ratio of the detection of analytes. Integration may be performed by increasing the collection time of the detector for the light arising from the array, and reducing the transfer rate of signal data out of the image sensor. To integrate analyte signal, a method of data transfer is provided using a CMOS active pixel sensor. A typical CMOS active pixel sensor is a fast frame rate device which may be used in video camera applications. In one embodiment, the CMOS active pixel sensor is operated in a far slower regime in order to integrate the array signal impinging on the sensor. In this embodiment, the integrated signal is stored in memory, the integration is repeated, and the rate of change of analyte signal over time is observed. The frame rate of the CMOS active pixel sensor may be controlled, for example, to integrate analyte detection by clearing all on-chip registers at time zero, and then collecting the analyte radiation signal for a fixed period of time. In some embodiments, individual photosensor elements of the image sensor perform integration simultaneously for different periods. Integration of the analyte signal increases its signal-to-noise ratio and enhances detection of analytes, allowing a lower concentration of analyte to be detected.

In one embodiment, analyte signal is enhanced by reducing the “dark current” noise inherent in the CMOS active pixel sensor by cooling the sensor within the low-light enclosure. The sensor may be cooled by a thermoelectric element, by nozzle expansion or refrigeration cooling methods, or by immersion in cooled fluids. A reduction of noise by about one-half is observed by cooling the sensor by 7° C., and cooling the sensor to 4° C. reduces noise by about ten-fold relative to room temperature. In some embodiments, a fluid, which may or may not contain sample molecules, is injected into the low-light enclosure to provide cooling for the sensor.

Analyte Array Signal

Optical detection of the analyte bound to a mesh probe includes detection by fluorescence, chemiluminescence, bioluminescence, and quantum dot methods. Label species or signalling molecules are attached to the polymer (resulting in a mesh reporter), or to the analytes in the target mixture. Examples of label species or signal molecules include radioisotopes, fluorescers, chemiluminescers, chemiluminophores, bioluminescers, enzymes, antibodies, and particles such as magnetic particles and quantum dots. Fluorescent dye molecules attached to a short amine-derivatized oligonucleotide may be used as a label species, where the amine group is coupled to a polymer. Signal molecules used for analyte detection include radiolabels, fluorescent dyes such as Cy3, Cy5, Alexa Fluor 488, fluorescein, rodamine, Texas red, rose bengal, dansyl chloride, ethidium bromide, aminonapthalenes, pyrenes, and porphyrins, chemiluminescent systems such as luminol, dioxetanes, acridinium phenyl esters, and ruthenium salts, chromophores and colorimetric probes such as colloidal gold, azo dyes, quinolines dyes, and cyanine dyes.

Examples of label species used include agonists and antagonists, toxins, epitopes, hormones, antibodies, peptides, enzymes, oligonucleotides, peptide-nucleic acids, lectins, carbohydrates, proteins and drugs. For example, enzymes used in ELISA assays may be used for fluorescence detection. Another example is fluorescent-labeled avidin or streptavadin.

In some embodiments, more than one type of label species is used to provide more than one method of detection for a particular analyte. The polymer of the mesh reporter may be coupled to a plurality of fluorescent and chemiluminescent label species, for example. As described above, in some embodiments, the mesh probe is capable of binding more than one target. Thus, in some embodiments, a polymer of the mesh reporter may be coupled to a plurality of target binding molecules and a plurality of different signalling species.

For fluorescence detection, array spot excitation light may be provided by an LED panel adjacent to the array, or alternatively adjacent to the CMOS sensor enclosure. In the fluorescence method, a narrow-band filter may be used adjacent to the array, between the array spots and the photodiodes to remove the excitation signal from the read out signals of the array, and to select the emitted light for detection.

In another method, analyte signal may be read out to provide assay information by optical detection of chemiluminescence. Chemiluminescence arises from light generated by a chemical reaction, which can be detected by a broadband detector without a filter, such as a CMOS active pixel sensor. Light from the array spot is detected directly, and the background signal is mainly due to “dark current.” In this instance, the mesh reporter is derivatized with streptavadin or anti-digoxigenin antibody, and chemiluminescent tags. Alkaline phosphatase or horse radish peroxidase, for example, can be used for chemiluminescence detection. The efficiency of detection may depend, in part, on the efficiency of attachment of the tags selectively or specifically to the targets. The label may be either biotin or digoxigenin that can be recognized by an enzyme detection system, followed by chemiluminescent reaction that converts the energy released from a chemical bond cleavage to photons of a discrete wavelength.

The ratio of the number of signal molecules or dye molecules to the number of binding molecules which are coupled to the backbone of the mesh reporter may be varied substantially. In some embodiments, the ratio of signal molecules to binding molecules is at least 3, 4, or 5. Often, the ratio of signal molecules to binding molecules is at least 6, 7, 8, or 9. Sometimes the ratio of signal molecules to binding molecules is at least 10, 20, 30, 40, 50, 60, 70, 80, 90, or 100. Various combinations of signal molecules and biding molecules may be used to form the mesh reporter.

In some embodiments of this invention, the array signal may be detected by a digital image sensor. In other embodiments, detection may be achieved with a charge coupled device (CCD), photomultiplier (PMT) or avalanche photodiode. Measurement of the analyte can also be done, for example, in various array schemes by electrical conductance detection.

Another aspect of the present invention relates to the application of mesh probes in industrial, environmental, biomedical and biotechnology fields. Mesh probes of this invention can be used in analytical or diagnostic applications, and to detect analytes in solution, gas or solid phase. The Mesh probes may be incorporated and used in a biosensor, to detect organic, inorganic or environmental particles in an analyte, or in an aqueous solution, non-aqueous phase, or gaseous phase.

Sensor System

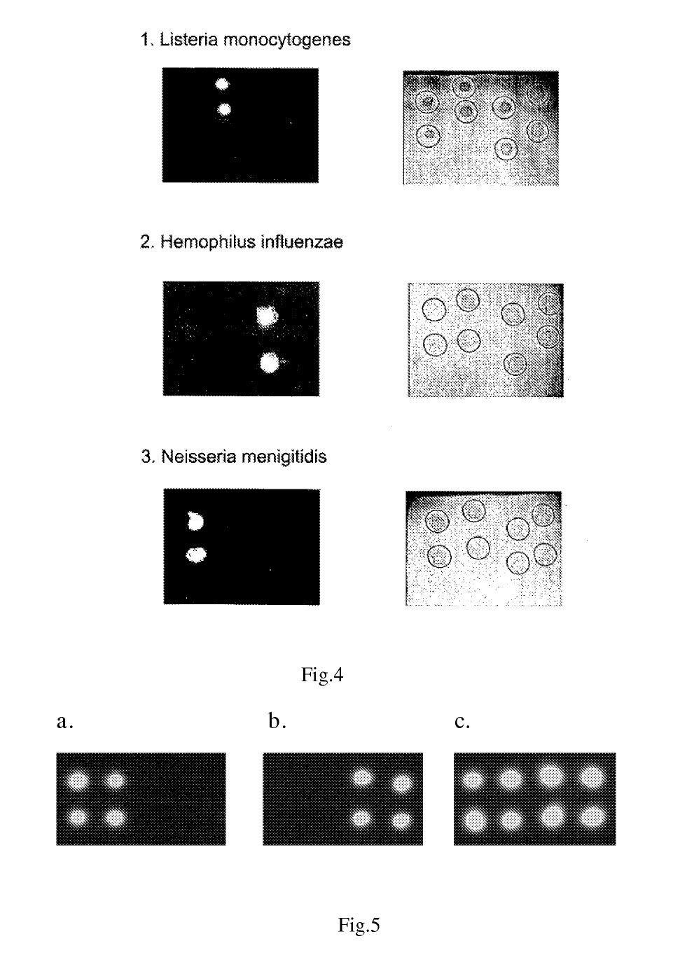

In one embodiment, as illustrated in FIG. 4, the biosensor system comprises a digital image sensor 600 in a low-light enclosure 100, a high data throughput reading station 400, and a general purpose computer 700. The reading station 400 has at least one socket for inserting a low-light enclosure 100 containing a CMOS digital image sensor 600, thereby electrically and mechanically connecting the sensor enclosure to the reading station. The reading station may be connected to the computer through universal serial bus (USB) 454, for example, or by a different parallel port interface device 452. Optionally, the reading station may be connected to the computer via Ethernet interface 456.

Referring to the embodiment of FIG. 4, a programmable logic device 460 on a motherboard in the reading station 400 is interfaced to a general purpose computer 700, such as a personal computer. The programmable logic device 460 is also interfaced to the digital image sensor 600, and synchronizes the read out of digital image sensor analyte data by monitoring status lines from the digital image sensor which signal the start and end of images, including frame, line, and pixel data clock pulse lines. The programmable logic device 460 manages the flow of image data out of the image sensor 600 and into local FIFO memory, where the image is stored until the computer 700 requests a transfer of the data. A typical cycle for the programmable logic device 460 is to receive a command from the computer 700, which causes the sensor to output an image, capture that image into local FIFO memory on the motherboard of the reading station 400, and transfer the captured image data to the computer 700. A graphical user interface (GUI) provides facility for the computer user to request an image capture and display cycle, a snap shot mode, or request a continuous sequence, a live video mode. Filters and image processing tools are provided to allow the user to operate the sensor under low-light conditions. These tools comprise image processing routines to boost small signals from the sensor, software routines to co-add images, routines to subtract background images or “dark” images, and routines to filter out noise. The GUI also gives the user control over the sensor on-chip settings. This allows the user to interact with the sensor, adjusting on-chip parameters such as integration time, gain, and analog to digital converter range.

The reading station includes a connector to attach the image sensor daughterboard and low-light enclosure to the reading station. A feature of this arrangement is that the digital image sensor analyte detector is readily mechanically separated from the reading station to provide high throughput operation of the biosensor system. A portable enclosure for a digital image sensor comprises the low-light enclosure. This plug-and-play feature of the biosensor system allows operation of the biosensor system with a portable enclosure, which is a disposable image sensor detector enclosure, for example. In alternative embodiments, the portable image sensor detector enclosures can be regenerated for use with a different array.

The reading station includes a USB microcomputer interface. Optionally, a MICROSOFT EXTENDED CAPABILITIES PORT (ECP) interface may be included, with a user controlled switch to determine the active interface. The USB cable supplies electrical power which can be used by the reading station motherboard. For ECP, a 9 VDC supply is provided. A manual reset switch is provided to reset the biosensor motherboard, and the programmable logic device may also be manually reset.

In one embodiment, this invention is a method of enhancing analyte signal detection by time-integration. Data throughput and measurement of analyte parameters in the target are limited by the signal-to-noise inherent in the detection of light from the array by the digital image sensor. The signal-to-noise may be increased by integrating analyte signal for several milliseconds or longer, often from about 10 milliseconds to about two minutes, sometimes about 30 to about one thousand milliseconds, and sometimes about 50 to about 600 milliseconds. In another embodiment, the time dependence of analyte signal is recorded by storing a sequence of array signal frames, in which each frame is obtained by integrating analyte signal for a period of time.

The USB microcomputer interface provides the master clock for the image sensor and programmable logic device. The image sensor output includes pixel data along with image line and frame pulses, which are passed back through the connector to the motherboard and sent into a FIFO memory. The frame pulse is used to reset the FIFO pointer, and the line pulse is used as the write enable for the FIFO. This arrangement stores pixel data in the FIFO starting with the upper left pixel as location 0 (zero) of the FIFO.

Once the image is in the FIFO, it can be read out by one of two interfaces. Referring to the embodiment of FIG. 4, ECP is provided in which the array image data is read into a parallel port (PP) 452, one pixel per read. The read starts when the PP 452 sends a reverse request. This causes the programmable logic device 460 to enable its output drivers to the PP 452. Then the programmable logic device 460 asserts the per.clock. The PP 452 responds with per.ack. The clock ack sequence continues until the computer 700 has read a frame of pixels. The programmable logic device 460 uses PP 452 data bit zero as SDA, and PP 452 data bit 1 as SCL of the I2C bus.

In another embodiment, image data in the FIFO is read out by USB interface. Biosensor operation is enhanced by increasing the bandwidth of serial data transmission as compared to conventional USB transfer. In conventional USB transfer, a packet of data from the FIFO would be read, followed by an interval of time in which the FIFO loads the next packet to be read out. For example, in a conventional USB microcomputer interface a packet of 63 pixels is read from the FIFO and sent via one of the data lines in the USB. In one embodiment, two end points are designated in the FIFO to establish two buffers. In operation, one buffer is read out and transmitted on one of the data lines in the USB while the other buffer is being filled, thereby increasing the transfer bandwidth using the universal serial bus by up to 100%. The end of the data transmission from the first buffer occurs immediately before, for example, one or a few clock pulses before, the start of data transmission on a data line of the universal serial bus from the second buffer. Then data transmission on a data line of the universal serial bus from the second buffer occurs, while at the same time loading data into the first buffer. These steps may be repeated until all the data in need of transfer is sent, thereby increasing the data transfer rate over conventional USB.

The following examples further describe embodiments of the present invention. The examples are given solely for the purpose of illustration and are not to be construed as limiting the present invention. While there have been described illustrative embodiments of this invention, those skilled in the art will recognize that they may be changed or modified without departing from the spirit and scope of this invention, and it is intended to cover all such changes, modifications, and equivalent arrangements that fall within the true scope of the invention as set forth in the appended claims.

All documents, publications, treatises, articles, and patents referenced herein are specifically incorporated by reference in their entirety.

### EXAMPLES

### Example 1

Linear polysaccharide dextran (Sigma) was dissolved in deionized water to a final concentration of 1% and then autoclaved. An aliquot of 0.40 ml dextran solution was oxidized with 44 microliter of 0.5 M sodium periodate overnight in the dark at room temperature on a rocking platform. The oxidized dextran was then cleaned by precipitation twice with 0.3 M NaOAC and 2×Vol of EtOH. The pellet was air-dried and redissolved in 0.4 ml of 5 mM NaP04 buffer, pH 7.2.

One microliter of the oxidized dextran was added to 7 microliters of 10 mM NaCO3 (pH 9.0) and 2 microliters oligonucleotides (2 uM solution in H20) in an Eppendorff tube. The oligonucleotides varied in length from 25 to 45 mer, with a primary amine introduced at either the 3′ or 5′ end during synthesis. The reaction was carried out overnight in a 37° C. water bath. NaBH4 was added to the tube and the mixture was incubated further for 30 minutes at room temperature, then precipitated with 0.3 M NaOAc and 2×Vol of EtOH. The pellet was dissolved in TE buffer and an aliquot was resolved on a 1% agarose gel by electrophoresis, and subsequently stained with EtBr. In this detection system, free oligonucleotide migrated close to the salt-front, while oligonucleotide coupled to dextran migrated much slower.

### Example 2

High-molecular-weight branched polysaccharides, glycogen (Sigma) and amylopectin (Sigma), were used to couple amine-derivatized oligonucleotides.

Diol groups of these polymers were converted to aldehyde groups by oxidation with NaI0₄. Sodium periodate was added to 0.4 ml of 1% polysaccharide solution (in H20) to a final concentration of 25 mM for glycogen, and 20 mM for amylopectin, respectively. Oxidation was continued in dark overnight at room temperature on a rocking platform. The oxidized polysaccharide was then precipitated twice with 0.3 M NaOAc and 2×Vol of EtOH to remove the excess NaI04. After air-drying, the pellets were dissolved in 0.4 ml of 5 mM NaP04 buffer (pH 7.2). Coupling of amine-derivatized oligonucleotide and gel-analysis of the coupled products were carried out as described in Example 1 for dextran.

### Example 3

The mesh probe of Example 2 is prepared having, on average, about 1000 oligonucleotide molecules coupled to each glycogen molecule at a coupling density of one oligonucleotide per 10 glucose monomers.

### Example 4

Signaling molecule 5′-ACTGCT-3′ (BP001) derivatized at the 5′ end with amine and at the 3′ end with fluorescent dye Cy5, and recognition probe molecule oligonucleotide AKH108 (5′-CCGTGCAGATCTTAATGTGCCAGTAAAAG-3 derivatized at the 5′ end with an amine group are coupled to the same polysaccharide. AKH108 hybridizes to a PCR product amplified with the primers of 5′-CCGTGCAGATCTTAATGTGC-3′ and 5′GCGCTGTACCAAAGGCATC-3′ from the bacterium Haemophilus influenzae genome, which corresponds to a fragment within the gene encoding 3-phosphoglycerate kinase. The PCR product is spotted onto a glass slide coated with poly (Lysine) in a microarray format.

For co-cross linking, 0.2 nmoles of AKH108 and 2 nmoles of BP001 are added to a tube containing 20 nmoles of oxidized glycogen in 10 mM NaCO3 with a final volume of 10 microliters. The reaction is carried out at 37° C. overnight.

Then NaBH4 is added to the tube to a final concentration of 4 mM and incubated at room temperature for another 90 minutes. The final products are precipitated with EtOH. After centrifugation, the cross-linked products as well as free AKH108 come down in the pellet, while free BP001 remains in the supernatant and is discarded. The pellet is dissolved 10 μl 3×SSPE/0.1% SDS/1.0 mg/ml BSA and is applied to the microarray surface. After hybridization at room temperature for five hours, the slide is washed 3 times with 10 u.l of fresh 0.1×XSSPE/0.1% SDS, and scanned in a laser scanner, and the spotted pattern for the PCR product is observed.

### Example 5

Oligonucleotide 5′-NH2-CCGATGCCTTAGTTTCAA-GTGGTGCGATTGACATCGTTGTCAT-3′, which specifically hybridizes to a PCR product amplified from the RecA gene from Enterococcous faecalis, was used to cross link to glycogen and amylopectin. The polysaccharides were oxidized as described in Example 2. For cross linking, 20 nmoles of the oxidized sugar and 2 nmoles of the amine derivatized oligonucleotides were mixed in 10 mM NaCO₃ buffer (pH 9.0) in a final volume of 10 microliter, and then incubated at 37° C. overnight. At the end of the coupling reaction, NaBH₄ was added to a final concentration of 4 mM and incubated for another 60 minutes at room temperature.

The final products were precipitated with 0.3 M NaOAc and 2×Vol of EtOH, then dissolved in 10 mM NaCO₃ (pH 9.0). Aliquots of the cross linked oligonucleotides were used to spot onto Epoxy treated glass slide. An equivalent amount of the oligonucleotides mixed with the unoxidized polysaccharide was also spotted onto the same slide as a control. For on-chip hybridization, the RecA PCR product was labeled with Alexa Fluor 546 (Molecular Probes Inc), dissolved in 3×SSPE/0.1% SDS/1.0 mg/ml BSA, and applied to the glass slide surface. After three hours hybridization at room temperature and washes with 0.1×SSPE/0.1% SDS, the slide was scanned using a laser scanner. The spot signal for the oligonucleotide coupled to glycogen was 5.86 relative to the control unoxidized glycogen spot, and the spot signal for the oligonucleotide coupled to amylopectin was 5.63 relative to the control unoxidized amylopectin.

### Example 6

A CMOS image sensor was used for direct on-chip detection of hybridization signals. The bare die of a PB0330 monochrome image sensor (Photobit) was attached to a daughter board with an edge connector. The bond wires were encapsulate in Epoxy and cured. The die surface was rinsed three times with autoclaved dH20 and air-dried. To derivatize the surface with epoxy, a solution of 2% (3-glycidoxypropyl) trimethoxy-silane in methanol was applied to the die surface and incubated at room temperature for 10 minutes, then washed twice with methanol and air-dried.

Four different capture probes (100 pmoles/ml in 50 mM NaCO3 buffer, pH 10.5) were manually spotted, in duplicate, onto the Epoxy treated die surface.

After the spots dried, the surface was quickly washed once with 1% ethanolamine in 50 mM NaCO₃ (pH 10.5), and incubated in the same blocking solution for 10 minutes at room temperature. The surface was then rinsed four times with autoclaved dH20.

For on-chip hybridization, the die surface was incubated with 3×SSPE/50% formamide/1 mg/ml BSA for 20 minutes at room temperature, then hybridized with a PCR product (biotin label at one end, as described below) in 20 microliter of 3×SSPE/50% formamide/1 mg/ml BSA (after it had been heated in a boiling water bath for 2 minutes and quickly cooled at 4° C.). The hybridization was carried out at 30° C. overnight in a moisturized chamber. Afterwards, the die surface was washed with 0.1×SSPE at room temperature four times, 5 minutes each. To bind streptavidin-alkaline phosphatase conjugate to the biotin on the hybridized PCR product, the die surface was first incubated with 1 mg/ml BSA in TBS at room temperature for 20 minutes, then incubated with Avidx-AP (Tropix) at 1:100 dilution in TBS/1 mg/ml BSA for two hours at room temperature. The die surface was then washed with TBS five times, 5 minutes each at room temperature.

For detecting the on-chip hybridization signal, the daughter board with TBS on the die surface was inserted into the connector on the Reading Station in a light-proof enclosure. A proprietary software was launched on a PC to retrieve a “dark image” (i.e. the background image, Idark). With the daughter board still attached to the Reading Station, the TBS was then replaced with a chemiluminescent substrate solution that include CDP-star and the enhancer Emerald II (both from Tropix) prepared according to the vendor's specifications.

An image frame (I) with about 0.1 sec integration time was retrieved from the sensor. I-I dark was treated as a real signal snapshot. The software has a built-in subroutine that adds successive processed snapshots together and displays the result as one image, thus further extending the signal integration time.

The following sensor register settings were found to be optimal at this point: Gains (Registers 53, and 43-46) at the maximum; Integration time (Registers 9 and 10) at 0.1 second per frame; Analogue negative offset (Registers 32 and 57) at the maximum; Gainstage (Register 62) at 74. The rest of the Registers were left at the default setting.

### Example 7

### Dopping Synthetic Polymer with TOTDA

Add to an Eppendorff tube 8 microliters of 1% oxidized Dextran-500 in 5 mM phosphate buffer (pH 7.2), 1 microliter of 1 mM 5′-amine derivatized oligonucleotide, 2 microliters of 0.2 M of Na2BO₃ (pH 9.0), 2 microliters of 20 mM NaBrH3CN, 1 microliter of 0.3 mM TOTDA, 6 microliter H2O. Mixed well and incubated overnight at 4° C., then added 4 microliters of H2O and 6 microliters DMSO, transferred to a microtiter plate, printed onto a chip surface on a GeneMachine OmniGridAccent arrayer. The ratio of the doping cross-linker verse that of the capture molecule can vary from 10:1 to 1:1000.

### Example 8

### Mesh Reporter with G-Quartet

A G-rich oligonucleotide TP (TGGACCAGACCAGCTATGGGGGAGCTGGGGAA GGTGGGAATGTGA) derived from the switch region of immunoglobulin gene has been shown to a four-stranded G4-DNA structure via Hoogsteen base-pairing between guanines (the underlined G), described in Sen and Gilbert (1988) Nature 334:364-366, Sen and Gilbert (1990) Nature 344: 410-414. A truncated version of TP, i.e. oligonucleotide Q3 (5′-CACGTATGGGGGAGCTGGGGTAT-3′), has been used to prepare G4-DNA affinity matrix for the purification of G4-DNA specific nuclease, described in Liu and Gilbert (1994) Cell 77: 1083-1092. Once formed in the presence of sodium or patassion ions, such four-stranded structure is very stable.

Add to an Eppendorff tube, 25 microliters of 2 mM HRP, 5 microliter of 0.3 mM Q3 oligo derivertized at the 5′-end with an amine, 10 microliters of 20 mM NaCNBrH3, 5 microliter of 0.1 mM straptavidine, 40 microliter of 1% oxidized Dextran-500, and 5 microliter of H2O. Incubate overnight a 4° C., then pass through a spin column made of Bio-Gel P-50 matrix. Collect the flow-through fraction and diluted 1:1 with a buffer containing 40% glycerol, 200 mM NaCl, and 100 mM KCl, stored at −20° C. This reagent can be used for detection of sample molecule containing biotin tag with enhanced sensitivity. Compared to direct polymerization of HRP, the poly-HRP prepared by the present method have several advantages. Because of there are more spacing between the HRP, this form of poly-HRP has less buoyant density, thus less likely to stick to the detection surface due to sedimentation, and consequently less non-specific binding noise signal. In addition, the better spacing between HRP will make it less likely to run into the problem of substrate depletion or the substrate diffusion become the rate-limiting step, which decrease the chemiluminescence signal generated. One can easily substitute the HRP with Alkaline phosphatase or a fluorescent dye to make other kind of mesh reporter.

The presence of Q3 oligo on the dextran backbone is mainly to provide mechanical stability to alleviate the problem of backbone breakage caused by mechanical shearing. It is well known that G-rich oligonucleotides can readily form, in the presence Na or K ion, a very stable four stranded structure (called G4 DNA or G-quartet), via Hoogsteen base-pairing.

The role Q3 oligo on the dextran is to provide certain degree of physical linking (or bridging), non-covalently, among the polymer backbones, which could be either intra or inter molecular in nature.

Of course, amine-derivatized Q3 oligonucleotide can also be used, e.g. replacing TOTDA described in Example 8, to prepare mesh probe, which will result in a stable, non-covalent physical linking of the polymer backbone, hence provide a better mechanical stability.

### Example 9

### Mesh Reporter Stabilized by Watson-Crick Base-Pairing

There are other means for non-covalent linking of the polymer backbones. One example is to utilized the specific base-pairing between complementary oligonucleotides. One can synthesis a 17-mer of random sequence and derivatized at the 3′-end with a amine group; then synthesis its complementary oligonucleotide and derivatized at the 3-end with amine. In combination, these two oligonucleotides can be used replace the Q3 oligonucleotide described in Example 9 in preparing the mesh reporter.

### Example 10

### Sensitivity Titration with Oligo Hybridization

One of the main reason to use mesh probe and mesh reporter in conjunction with a direct probe-on-sensor approach is to achieve a greatly improved sensitivity. The following is an example to assess the sensitivity of this combined system.

Two different oligonucleotides, RHB101 and NHY205, are cross-linked to oxidized dextran separately as described in Example 6. Each mesh probe was printed onto a CMOS sensor chip in a 2 by 2 subarray. A total of seven chips were printed. After the printing, the chips were let dry in a 70% humidifying chamber for two hours, then baked in a 80° C. vacuum oven overnight. The chips were then wash with Solution I, then followed by washing with Solution II. Hybridization solution containing the biotinylated complementary oligos, BCRHB101 and BCNHY205, were added to the chips. For all seven chips, the amount of BCRHB101 added was held at constant of 10-17 moles per chip (or hybridization), while that for the oligo BCNHY205 was decreased by 10 folds for each chip, varying from 10-16 to 10-21 moles. For the seventh chip, BCNHY205 was omitted from the hybridization reaction. After the hybridization, the chips were thoroughly washed with 0.1×SSPE, and then incubated with Straptavidin-PolyHRP in TBS buffer containing 1% BSA for an hour. After this incubation, the chips were washed with TBS several times, and then the chemiluminescent substrate for HRP was added to the chips. The hybridization results were retrieved on a desktop personal computer through a USB Reading Station.

Several set of data consistently showed that the sensitivity of this system is about 500 molecules (i.e. total added, which includes the free as well as the bound species after the hybridization). In the absence of BCNHY205, there was no hybridization signals on the “spots” of NHY205 mesh probe. To assess the dynamic range of the system, the hybridization signals from NHY205 were divided by or normalized with those from RHB101, then plotted against the concentration of BCNHY205. On a double-log plot, the linear range appears to at least 4.5 log.

### Example 11

### Non-Amplified Detection of a Gene from Buffy Coat

Ten milliliters of blood is collected from a human patient and spun down in a centrifuge. The white layer of “Buffy Coat” cells are drawn from the centrifuge tube, and add to a fifteen milliliter centrifuge tube containing three milliliters fine glass beads and six milliliters PBS buffer. The tube is then sonicated on a tabletop sonicater for one minute, then repeat four more times. Twenty microliters of the crude lysate is removed from the tube and add to a CMOS sensor chip containing a capture probe for human CYP 450 gene, then add one microliter of reporter probe that is end-labelled with a biotin tag and hybridizes to a sequence adjacent to that of the capture probe. After two hours of hybridization, the chip is wash under a stringent condition. Mesh reporter containing Straptavidin and poly-HRP is then added and incubated for an hour at room temperature. The chip is washed again for several times with PBS buffer, and the chemiluminescent substrate (LMA-6, Lumigen) is added to the chip. The hybridization result is retrieved on a laptop computer through a portable USB reading station.

There are about twelve millions white blood cells in ten milliliters blood. Assuming one is examining a specific haplotype of CYP 450, and a total sample recovery of seventy percent after going through the procedure described above, one would have about 10,000 copies CYP gene in the twenty microliter crude lysate that was used for the on-chip hybridization assay. The current system is more than sensitive enough to detect the specific hybridization event, hence bypass the need for a prior enzymatic amplification of the nucleic acid target sequence.

### Example 12

### Non-Amplified Detection of Micorbial Pathogens Based

Ribosomal RNAs is an abandant nucleic acid species in living cells, estimated to be around 10,000 molecules per cell. Though these RNAs are quite conserved in the nucleotide sequence, they do have organism-specific variations in the sequences that are sufficient for a definitive identification of a organism by specific nucleic acid hybridization. The following is an example for microbial pathogen detection.

To illustrate the detection principle in general, a solution of Chlamydia trachomatis (CT) at the concentration of 10,000 cfu/ml is used. A 0.3 milliliter aliquot of the CT solution is mixed in an Eppendorff tube with 0.3 milliliter of PBS buffer containing 2% SDS and 0.3 milliliter (volume) of glass beads, then vortexed at the top speed with a bench top Vortexer. An aliquot of 10 microliter of the supernatant is mixed with 10 microliter of solution containing 90% formamide and 6×SSPE, plus 1 microliter of biotinylate reporter probe, then transferred to the sensor chip that has corresponding capture probe tethered on the surface via polymer backbone and hybridized for an hour. Both the reporter and capture probes hybridize to the same ribosomal RNA molecule in proximity under a defined hybridization conditions. They range in size from 17 to 45 nucleotides. After the hybridization, the chip is processed as described in above examples. To do this kind of tests in a clinic setting, it is desirable to include on the chip other capture probes for both positive and negative controls. Also, one may include the capture and reporter probes for other pathogens, such as Neisseria gonorrhoeae (NC), in the same assay.

Assuming the yield of CT cell lysis with glass bead-vortexing is about 10%, a calculation indicates that, for any given ribosomal RNA species, the estimated target molecule present in each hybridization reaction as described above is about 150,000 as the lower bound, which is more than sufficient for detection by this system without any prior enzymatic amplification.

To test for CT/NC in a clinic setting, the common pathogens of sexually transmitted disease, the vaginal swab is rinsed in PBS buffer. The washed off cells are spun down, then resuspended in 1 milliliter PBS. For CT/NC positive patient, the respective cells collected in such manner will be much higher than 10,000 cells/ml. The assay can be carried out as described above from this point on. The principle disclosed can also be applied to non-amplified detection of water-borne and food-borne microbial pathogens.

### Example 13

The cover slip (3.6×3.6 mm) was soaked in 1M NaOH and shook for 2 hours, then the slips were rinsed two times with deionized water and nitrogen-dried. The surface was derivatized with (3-glycidoxypropyl)trimethoxy-silane overnight in a vacuum-desiccator, and then baked in a 80° C. vacuum oven for three hours.

The capture antibody of AFP or PSA was dissolved in 1×PBS/20 mM NaBO₃/5% Glycerol buffer (pH8.0) to a final concentration of 0.3 mg/ml. Each antibody was printed onto a cover slip in a 2 by 2 sub-array. After the printing, the slips were let dry in a 70% humidifying chamber for two hours, and then preserved in a 4° C. vacuum-desiccator.

The slips were soaked in 1×PBS/1% Ethanolamine (pH8.4) for 30 min, and then washed two times with washing buffer (1×TBS/5% Glycerol/0.1% BSA), followed by blocking the surface for 30 min with blocking buffer (1×TBS/5% Glycerol/1% BSA). AFP (5 ng/ml) and PSA (0.5 ng/ml) were added to the slips, separately or together, and incubated on a rocking platform for 1 hour at room temperature. After washed three times, the slips were correspondingly incubated with biotinylated detection antibodies of AFP and/or PSA (Tianjian Biotechnologies) diluted 1:10,000 in blocking buffer in the same condition. Then the slips were incubated with streptavidin-HRP (sigma) diluted 1:2,000 in blocking buffer for 30 min on a rocking platform and washed 5 times.

For detecting the hybridization signal, the slip, with TBS on the top, was directly attached onto the die surface of CMOS sensor. And then the sensor was inserted into the connector on the Reading Station in a light-proof enclosure. A proprietary software was launched on a PC to retrieve a “dark image” and get background. About one minute after the TBS was replaced with a ECL chemiluminescent substrate solution, the signal image was retrieved from the sensor.

1. A kind of molecule detecting system, wherein the said system comprising:
an optical sensor; and
an array of capture molecules which are attached on the sensor surface through cross linking to a polymer that has limited interconnects amongst the polymer chains.

2. The kind of molecule detecting system according to claim 1, wherein said array of capture molecules are attached on the surface of a thin transparent solid or porous substrate through cross linking to a polymer that has limited interconnects amongst the polymer chains, and the said transparent substrate is fixed above the surface of an optical sensor.

3. The kind of molecule detecting system according to claim 1, wherein said polymer is a natural polymer.

4. The kind of molecule detecting system according to claim 3, wherein said natural polymer is a linear or branched polysaccharide.

5. The kind of molecule detecting system according to claim 4, wherein said linear polysaccharide is dextran.

6. The kind of molecule detecting system according to claim 4, wherein said branched polymer is a glycogen or amylopectin.

7. The kind of molecule detecting system according to claim 3, wherein said polymer is branched-DNA.

8. The kind of molecule detecting system according to claim 3, wherein said polymer is a hydrogel.

9. The kind of molecule detecting system according to claim 1, wherein said capture molecule is a biomolecule.

10. The kind of molecule detecting system according to claim 9, wherein said biomolecule is a protein.

11. The kind of molecule detecting system according to claim 9, wherein said biomolecule is a DNA or RNA.

12. The kind of molecule detecting system according to claim 1, wherein said capture molecule of claim is a PNA or LNA.

13. The kind of molecule detecting system according to claim 1, wherein said polymer is a synthetic polymer.

14. The kind of molecule detecting system according to claim 13, wherein said synthetic polymer is Poly(methyl vinyl ether-alt-maleic anhydride)

15. The kind of molecule detecting system according to claim 1, wherein said interconnects amongst the polymer chains is covalent formed by cross-linking with a bi-functional cross-linker.

16. The kind of molecule detecting system according to claim 15, wherein said bi-functional cross-linker is 4,7,10-Trioxa-1,13-tridecanediamine.

17. The kind of molecule detecting system according to claim 1, wherein said interconnect amongst is form by non-covalent interactions.

18. The kind of molecule detecting system according to claim 17, wherein said non-covalent interaction is a four stranded DNA or RNA structure formed by Hoogsteen base-pairing between G-rich oligonucleotides cross-linked to the polymer.

19. The kind of molecule detecting system according to claim 18, wherein said G-rich oligonucleotide is Q3 derivatized at the end with a functional group for cross-linking to the polymer.

20. A method of making polymer used in molecule detecting system comprising following steps:
a) reacting a polysaccharide with sodium periodate, thereby forming a linear or branched polymer having a large number of aldehyde groups;
b) adding a molecule having or derivatized with a reactive amino groups; and
c) completing the coupling reaction in the presence of NaCNBrBH3, wherein the said polymer has limited interconnects amongst the polymer chains.

21. The method of making polymer used in molecule detecting system according to claim 20, wherein a reporter molecule of which both the signaling molecules and at least a recognition molecule are cross-linked to the polymer prepared.

22. The method of making polymer used in molecule detecting system according to claim 21, wherein said reporter which the signaling molecule is either horse reddish peroxidase or alkaline phosphotase and the recognition molecule is straptavidin.

23. A method for analyte detection comprising:
a) contacting an analyte sample with the array of capture molecules which are attached on the optical sensor surface through cross linking to a polymer that has limited interconnects amongst the polymer chains;
b) then contacting the array of molecule with the reporter of claim 21;
c) adding to the array a chemiluminescent substrate;
d) retrieving the data directly from the optical sensor without the use of an external scanner.

24. The method for analyte detection according to claim 23, wherein said analyte is a nucleic acid, and the analyte sample has not been subject to any enzymatic amplification.

25. An array of capture molecules for use in detecting analytes on an optical sensor, where the capture molecules are attached on the surface of a thin transparent solid or porous substrate through cross linking to a polymer that has limited interconnects amongst the polymer chains, and the said transparent substrate is fixed above the surface of an optical sensor.
